(19) United States
(12) Patent Application Publication
(10) Pub. No.: US 2003/0026766 A1
(43) Pub. Date: Feb. 6, 2003

(54) Medicaments for treating respiratory disorders comprising formoterol and fluticasone

(76) Inventor: Mark Sanders, Bedforshire (GB)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/009,956

(22) PCT Filed: Apr. 12, 2001
(86) PCT No.: PCT/GB01/01656

(30) Foreign Application Priority Data
Apr. 13, 2000 (GB) 0009046.4
Mar. 10, 2001 (GB) 0105967.4

(51) Int. Cl. A61L 9/04; A61K 9/14

(52) U.S. Cl. 424/46

(57) ABSTRACT

There is described a method of treating or alleviating a respiratory disorder which comprises administering an effective amount of the active ingredients formoterol, or a pharmaceutically acceptable salt thereof, and fluticasone, or a pharmaceutically acceptable ester thereof, separately, sequentially or simultaneously, provided that the active ingredients comprise separate compositions. There is also described a dry powder inhaler containing formoterol, or a pharmaceutically acceptable salt thereof, and fluticasone, or a pharmaceutically acceptable ester thereof, which may be administered separately, sequentially or simultaneously, provided that they arm administered as separate compositions

This invention relates to a novel method of treatment and to a novel use of known medicaments.

Formoterol or N-[2-hydroxy-5-[1-hydroxy-2-[[2-(4-methoxyphenyl-)1-methylethyl]amino]ethyl]-phenyl]formamide is known from British Patent No 1415256. Formoterol is a β-adrenoreceptor agonist which has antiasthmatic properties and selective bronchodilator properties.

Fluticasone or S-fluoromethyl 6α, 9α-difluoro-11β-hydroxy-16α-methyl-17α-hydroxy-3-oxoandrosta-1,4-diene-17β-carbothioate is an anti-inflammatory corticosteroid with minimal liability to undesired systemic side effects which is described in British Patent No 2089877.

Numerous attempts have been made at preparing efficacious combination therapies. Thus, a combination therapy of fluticasone, i.e. fluticasone propionate, and a bronchodilator, namely salmeterol, is known from U.S. Pat. No. 5,270,305. Furthermore, European Patent Application No. 9202826 describes formoterol and budesonide combinations and European Patent No 0 416 951 describes salmeterol and fluticasone combinations.

However, each of these combination therapies suffers from certain disadvantages, inter alia, they may be unsuitable for use in the treatment or alleviation of acute asthma symptoms or may not be optimal for the treatment of the inflammatory component of the disease.

More recently, International Patent Application No. WO 00/48587, Clarke et al, which is an intervening publication, published on Nov. 1, 2000, describes a pharmaceutical composition comprising formoterol fumarate and fluticasone propionate which as being useful in the treatment of inflammatory or obstructive airways disease.

We have now surprisingly found that a combination of formoterol, or a salt thereof, and fluticasone, or an ester thereof, can be therapeutically effective if the medicaments are administered separately, sequentially or simultaneously, provided that such administration comprises separate compositions of the two active ingredients. The administration of a combination of fluticasone, or a pharmaceutically acceptable ester thereof, and formoterol, or a pharmaceutically acceptable salt thereof, separately, sequentially or simultaneously is advantageous in that it is more efficacious than other prior art combination therapies.

Thus, according to the invention we provide a method of treating or alleviating a respiratory disorder which comprises administering an effective amount of the active ingredients formoterol, or a pharmaceutically acceptable salt thereof, and fluticasone, or a pharmaceutically acceptable ester thereof, separately, sequentially or simultaneously, provided that the active ingredients comprise separate compositions.

According to a fiber embodiment, the method of the invention comprises the separate or sequential administration of formoterol, or a pharmaceutically acceptable salt thereof, and fluticasone, or a pharmaceutically acceptable ester thereof.

In an alternatively preferred embodiment the method of the invention comprises the separate administration of formoterol, or a salt thereof, and fluticasone, or an ester thereof.

In an especially preferred embodiment the method of the invention comprises the sequential administration of formoterol, or a salt thereof, and fluticasone, or an ester thereof.

In an alternatively preferred embodiment the method of the invention comprises the separate administration of formoterol, or a salt thereof, and fluticasone, or an ester thereof.

When the method of the invention comprises the sequential administration of the active ingredients, it is preferred that the method comprises the administration of formoterol, or a salt thereof, followed by the sequential administration of fluticasone, or an ester thereof.

The method of the invention is most advantageous in the treatment of respiratory disorders such as asthma and/or chronic obstructive pulmonary disease (COPD).

In the method of the invention the formoterol, or a salt thereof, and the fluticasone, or an ester thereof, may be administered in a variety of ways but the most preferred method of administration is by way of inhalation. Thus, the method of the invention may comprise administration by way of an inhaler, e.g. a metered dose inhaler or a dry powder inhaler, an insufflator, a nebuliser or any other conventionally known method of administering inhalable medicaments.

When administered by way of inhalation the method of the invention may comprise the use of a pressurised aerosol.

Thus, according to a further feature of the invention we provide a method which comprises administration by way of a pressurised aerosol comprising, separately, formoterol, or a salt thereof, and formoterol, or an ester, as hereinbefore described, each being in admixture with at least a suitable propellant and optionally with a surfactant or a mixture of surfactants. The propellant is preferably a non-CFC propellant, such as a hydrofluoroalkane (HFA). Any conventionally known HFA propellant may be used, including those disclosed in, for example, EP0372777, WO91/04011, WO91/11173, WO91/11495 and WO91/14422. However, the most preferred HFA is a fluoroalkane such as a fluoromethane or a fluoroethane or a mixture of fluoroalkanes. Such fluoroalkanes include, but are not limited to, trichlorofluoromethane, dichlorodifluoromethane, 1,2-dichlorotetrafluorethane, tichlorotrifluoroethane and chloropentafluoroethane. The most preferred is HFA 134 (1,1,1,2-tetrafluoroethane) or HFA 227. The amount of propellant present may vary, but generally the active ingredient to propellant ratio will be from 1 to 300 to 1 to 5. Mixtures of propellants may also be used, for example, a mixture of HFA 134 and HFA 227. Thus the aerosol compositions of the invention may be as a solution or a suspension each of the active ingredients with a propellant.

The pressurised aerosol formulations of the invention may be administered in any conventionally known inhalation apparatus.

In another embodiment the method may comprise administration of the active ingredients as dry powder formulations. Thus, according to the invention we provide a method as hereinbefore described which comprises administration by way of a dry powder inhaler wherein the inhaler comprises, separately, formoterol, or a salt thereof, and fluticasone, or an ester thereof, each, optionally in admixture with a suitable adjuvant, diluent or carrier.

The dry powder formulations of the invention may be administered in any conventionally known inhalation apparatus. However, such a dry powder inhaler comprising, separately, formoterol, or a salt thereof, and fluticasone, or an ester thereof, is novel per se.

Thus, according to a further feature of the invention we provide a dry powder inhaler containing formoterol, or a pharmaceutically acceptable salt thereof, and fluticasone, or a pharmaceutically acceptable ester thereof.

Each of the active ingredients may optionally be in admixture with a pharmaceutically acceptable adjuvant, diluent or carrier.

Any conventionally used ingredients in dry powder formulations may be used, as suitable adjuvant, diluent or carrier such as sugars, these include, but are not limited to, dextran, mannitol and lactose, e.g. α-lactose monohydrate. Preferably, the active ingredient to carrier ratio is from 0.001:1 to 50:1, for example, 0.4% w/w.

In a dry powder inhaler the formoterol, or a pharmaceutically acceptable salt thereof, and the fluticasone, or a pharmaceutically acceptable ester thereof, may be administered separately, sequentially or simultaneously, provided that the active ingredients comprise separate compositions.

Preferred dry powder inhalers are those described in our co-pending Patent application No. PCT/GB 00/03377 or PCT/GB 00/04623.

Alternatively, the formulations may be administered by way of a conventional nebuliser. A suitable nebuliser formulation consists of a sterile, isotonic solution of the pharmaceutical compositions of the invention in water, optionally containing one or more surfactants or a pharmaceutically acceptable co-solvent. Alternatively, the nebuliser formulation may comprise a suspension of the pharmaceutical compositions of the invention in finely divided form in a sterile isotonic solution. The solution or suspension may be nebulised by an air jet, dropping onto an ultrasonic vibrating plate, forcing through small orifices or other known types of nebuliser, including unit-dose nebulisers, including those described by Dolovich, M., “New Propellant-free Technologies under Investigation”, J. Aerosol Medicine, 1999; 12 (suppl 1): S9-S17, such as, Respimat (from Boehringer Ingelheim), AERx™ (from Aradigm), and AeroDose (from Aerogen).

For inhalation therapy the active ingredients are preferably micronised or reduced in size by other recognised mechanisms, such as spray drying, co-milling, etc. The particle size of the fluticasone, or a pharmaceutically acceptable ester thereof, and the formoterol, or a pharmaceutically acceptable salt thereof, may be the same or different. However, it is preferred that both fluticasone, or a pharmaceutically acceptable ester thereof, and formoterol, or a pharmaceutically acceptable salt thereof, will have an aerodynamic particle size of from 1 to 10 microns.

The dosage of each of the active ingredients administered to a patient may vary depending, inter alia, upon the nature and severity of the disorder being treated and the method of administration.

In a preferred embodiment, each metered dose or actuation of an inhaler will generally contain from 3 μg to 50 μg of formoterol, or a pharmaceutically acceptable salt thereof, and from 20 μg to 500 μg of fluticasone, or a pharmaceutically acceptable ester thereof. The frequency of administration of each of the active ingredients may vary, but most preferably, each of the active ingredients will be administered, separately, sequentially or simultaneously, but as separate compositions, once or twice daily, although other treatment regimes may be applicable.

According to a further feature of the invention we provide a method of treating COPD which comprises administering to a patient suffering from such a disorder a therapeutically effective amount of formoterol, or a pharmaceutically acceptable salt thereof, and formoterol, or a pharmaceutically acceptable ester thereof, separately, sequentially or simultaneously, provided that if the active ingredients are administered simultaneously, they are as separate compositions.

We also provide the use of fluticasone, or a pharmaceutically acceptable ester thereof, in the manufacture of a medicament for use in the method as hereinbefore described.

We further provide the use of formoterol, or a pharmaceutically acceptable salt thereof, in the manufacture of a medicament as hereinbefore described.

We also provide the use of formoterol, or a salt thereof, and fluticasone, or an ester thereof, in the manufacture of a dry powder inhaler as hereinbefore described.

According to a further feature of the invention we provide the use of formoterol, or a pharmaceutically acceptable salt thereof, and fluticasone, or a pharmaceutically acceptable ester thereof, as active ingredients in the manufacture of a medicament to be administered separately, sequentially or simultaneously, provided that the active ingredients comprise separate compositions for the treatment or alleviation of a respiratory disorder.

It is known that glucocorticoids are used for the suppression of inflammation in chronic inflammatory diseases which are associated with an increase in the expression of inflammatory genes (cytokines, enzymes, receptors and adhesion molecules). This is thought to be due in part to a direct inhibitory interaction between activated glucocorticoid receptors and activated transcription factors which results in regulation of the inflammatory gene expression. In this mechanism the inhibitory effect of the glucocorticoid on cytokine synthesis is considered to be of particular importance. It has also been found that glucocorticoids increase the expression of β₂ adrenoreceptors by increasing the rate of transcription of the human β₂ receptors.

Thus known combination therapies can be expected to be efficacious, but we have surprisingly found that the new therapy of the invention is especially advantageous in that tests indicate, inter alia, a significant increase in glucocorticoid receptor translocation to the nucleus and in immunocomplex formation.

Therefore according to a yet Her feature of the invention we provide a method of attaining improved glucocorticoid receptor translocation into the nucleus (and the functional consequences, for example on cytokine expression) by the administration of a therapeutically effective amount of a β₂ agonist and a steroid in therapeutically effective amounts wherein the method provides an improvement of at least 20%, preferably at least 35%, over prior art β₂ agonist and a steroid combination therapies.

In this particular feature of the invention the preferred method comprises the administration of therapeutically effective amounts of formoterol and fluticasone. The method may comprise an improvement of from 35-50% over known combination therapies.

Thus when measured as a change in density on a Western Blot strip, the method of this aspect of the invention may provide a percentage change in band density of at least 255, preferably of at least 300, for example, between 300 and 400 percentage change in band density.

This particular aspect of the invention is advantageous in that it may be useful in providing more efficacious therapies in a variety of inflammatory disorders, for example, asthma, rheumatoid arthritis, inflammatory bowel disease and autoimmune diseases.

According to a further feature of the invention we provide the use of a glucocorticoid, e.g. fluticasone, in the manufacture of a medicament with improved β₂ receptor expression.

In this aspect of the invention the improved β₂ receptor expression may be an improvement of at least 20% over prior art medicaments, preferably at least 35%, for example, from 35-50%.

Thus when measured as a change in density on a Western Blot strip, we provide the use of a glucocorticoid in the manufacture of a medicament with improved β₂ receptor expression measured as a percentage change in band density of at least 255, preferably of at least 300, for example, between 300 and 400 percentage change in band density.

The ratio of formoterol, or a pharmaceutically acceptable salt thereof, to fluticasone, or a pharmaceutically acceptable ester thereof, in the method of the invention may vary, but is preferably within the range from 1:0.4 to 1:167.

Suitable pharmaceutically acceptable salts of formoterol include acid addition salts derived from inorganic and organic acids, such as the hydrochloride, hydrobromide, sulphate, phosphate, maleate, tartrate, citrate, benzoate, 4-methoxybenzoate, 2- or 4-hydroxybenzoate, 4-chlorobenzoate, p-toluenesulphonate, methanesulphonate, ascorbate, salicylate, acetate, fumarate, succinate, lactate, glutarate, gluconate, hydroxynaphthalenecarboxylate e.g. 1-hydroxy- or 3-hydroxy-2-naphthalenecarboxylate, or oleate. The fumarate salt is especially preferred.

The formoterol, or a pharmaceutically acceptable salt thereof, may be present either as a racemic mixture, as a mixture of enantiomers or substantially as a single D- or L-isomer.

Suitable pharmaceutically acceptable esters of fluticasone include alkanoates, e.g. C₁ to C₁₀ alkanoates, preferably C₁ to C₅ alkanoates. The propionate ester is especially preferred.

The invention will now be described by way of example only and with reference to the accompanying drawings in which references to fluticasone are to fluticasone propionate and references to formoterol are references formoterol fumarate.

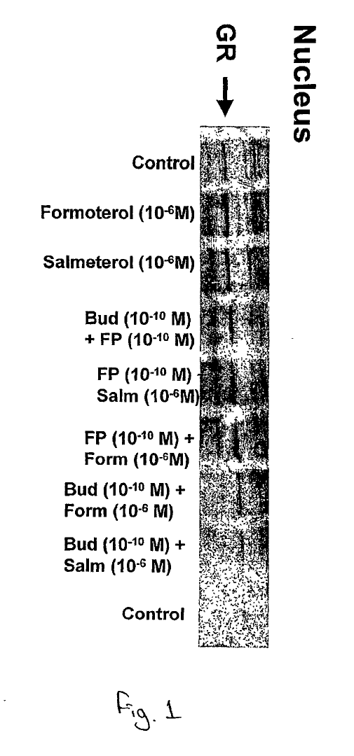

FIG. 1 is a representation of Western Blot strip following the assay of Example 1; and

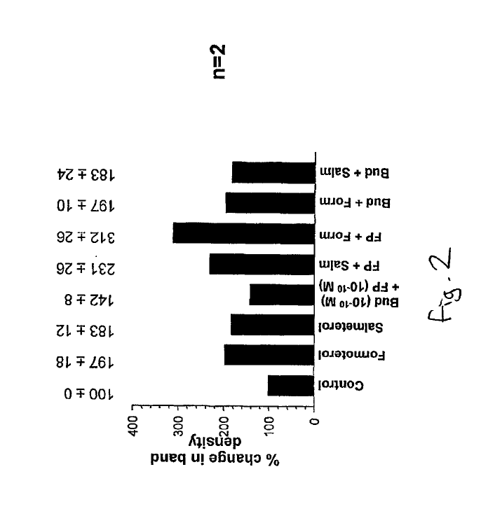

FIG. 2 is a bar chart based on the Western Blot of FIG. 1.

### EXAMPLE 1

Western Blot Analysis

Nuclear and cytosolic proteins wore extracted from U937 cells by gentle detergent lysis. Cells were lysed for 15 minutes at 4° C. using 0.1% NP-40 and cytoplasmic proteins collected. Soluble nuclear extracts were obtained following osmotic lysis (0.42 M NaCl) of the nuclear envelope. At least 20 μg/lane of whole-cell proteins were subjected to a 10% SDS-polyacrylamide gel electrophoresis, and transferred to nitrocellulose filters (Hybond-ECL, Amersham Pharmacia Biotech, Amersham, UK) by blotting. Filters were blocked for 1 h at room temperature in Tris-buffered saline (TBS), 0.05% Tween 20, 5% non-fat dry milk. The filters were then incubated with rabbit anti-human GR antibody (Santa Cruz Biotechnology, Santa Cruz, Calif.) for 1 h at room temperature in PBS, 0.05% Tween 20, 5% non-fat dry milk at dilution of 1:1000. Filters were washed three times in PBS, 0.05% Tween 20 and after incubating for 45 minutes at room temperature with anti-rabbit antibody conjugated to horseradish peroxidase (Dako, Ely, UK) in PBS, 0.05% Tween 20 and 5% non-fat dry milk, at dilution of 1:4000. After further three washes in PBS with 0.05% Tween 20 visualisation of the immunocomplexes was performed using ECL (see FIG. 1) as recommended by the manufacturer (Amersham Pharmacia Biotech).

The bands, which were visualised at approximately 94 kDa, were quantified using a densitometer with Grab-It and GelWorks software (UVP, Cambridge, UK) (see FIG. 2). The percentage change in band density is therefore proportional to increase in glucocorticoid receptor translocation into the nucleus

The results are given in Table 1.

TABLE 1

| | % Change in Band |
|---|---|
| Composition | Density |
| Control | 100 ± 0 |
| Formoterol | 197 ± 18 |
| Salmeterol | 183 ± 12 |
| Budesonide/Fluticasone | 142 ± 8 |
| Salmeterol/Fluticasone | 231 ± 26 |
| Formoterol/Fluticasone | 312 ± 26 |
| Formoterol/Budesonide | 197 ± 10 |
| Salmeterol/Budesonide | 183 ± 24 |

### EXAMPLE 2

Oedema Model Studies

Tests were performed to determine the effect of formoterol and fluticasone on the inhibition of lung inflammation. The test model employed was the Sephadex-induced oedema model.

Sephadex was administered intratracheally to Sprague-Dawley rats together with saline (control), formoterol, fluticasone, salmeterol, formoterol-fluticasone combinations, budesonide-fluticasone combinations, fluticasone-salmeterol combinations, budesonide-formoterol combinations and budesonide-salmeterol combinations. Animals were subjected to each relevant experimental regimen and were then sacrificed, their lungs excised and the inflammatory process measured as lung weight increase due to oedema.

The weight increase of lungs removed from animals subjected to the Sephadex-saline regimen compared to the weight of lungs removed from a second group of control animals, to which only saline was administered and this taken as maximum Sephadex induced oedema.

Inhibition of the Sephadex induced lung oedema by a test substance was determined as a percentage reduction of induced oedema in the presence of the test compound compared to the maximum oedema induced in the Sephadex-saline controls.

### EXAMPLE 3

Separate/Sequential Administration of Formoterol and Fluticasone

The experiments of Examples 1 and 2 were repeated using a dosing regimen comprising the separate and/or sequential administration of formoterol and fluticasone and experiments were extended to include determination of the functional consequence of the increase in receptor translocation on pro- and anti-inflammatory cytokine expression, including TNF alpha, interleukin 10, GM-CSF and interleukin 1-receptor antagonist.

1. A method of treating or alleviating a respiratory disorder which comprises administering an effective amount of the active ingredients formoterol, or a pharmaceutically acceptable salt thereof, and fluticasone, or a pharmaceutically acceptable ester thereof, separately, sequentially or simultaneously, provided that the active ingredients comprise separate compositions.

2. A method according to claim 1 characterised in that the formoterol, or a pharmaceutically acceptable salt thereof, and the fluticasone, or a pharmaceutically acceptable ester thereof, are administered separately or sequentially.

3. A method according to claim 2 characterised in that the formoterol, or a pharmaceutically acceptable salt thereof, and the fluticasone, or a pharmaceutically acceptable ester thereof, are administered sequentially.

4. A method according to claim 3 characterised in that the method comprises the administration of fluticasone, or a pharmaceutically acceptable ester thereof, followed by the sequential administration of formoterol, or a pharmaceutically acceptable salt thereof.

5. A method according to claim 2 characterised in that the formoterol, or a pharmaceutically acceptable salt thereof, and fluticasone, or a pharmaceutically acceptable ester thereof, are delivered separately.

6. A method according to claim 1 characterised in that the formoterol, or a pharmaceutically acceptable salt thereof, and fluticasone, or a pharmaceutically acceptable ester thereof, are administered by inhalation.

7. A method according to claim 6 characterised in that the formoterol, or a pharmaceutically acceptable salt thereof, and the fluticasone, or a pharmaceutically acceptable ester thereof, are administered by way of pressurised aerosols comprising a pharmaceutical composition in admixture with at least a suitable propellant.

8. A method according to claim 7 in which a surfactant is present.

9. A method according to claim 8 in which a surfactant is absent.

10. A method according to claim 9 characterised in that the surfactant is a mixture of surfactants.

11. A method according to claim 7 characterised in that the propellant, or mixture of propellants, is a non-CFC propellant.

12. A method according to claim 11 characterised in that the propellant, or mixture of propellants, is selected from hydrofluoroalkanes (HFA).

13. A method according to claim 12 characterised in that the propellant is HFA 134.

14. A method according to claim 12 characterised in that the propellant is HFA 227.

15. A method according to claim 12 characterised in that the propellant is a mixture of HFA 134 and HFA 227.

16. A method according to claim 6 characterised in that the formoterol, or a pharmaceutically acceptable salt thereof, and the fluticasone, or a pharmaceutically acceptable ester thereof, are administered by way of a dry powder inhaler.

17. A dry powder inhaler containing formoterol, or a pharmaceutically acceptable salt thereof, and fluticasone, or a pharmaceutically acceptable ester thereof, which may be administered separately, sequentially or simultaneously, provided that they are administered as separate compositions.

18. A dry powder inhaler according to claim 15 comprising formoterol, or a pharmaceutically acceptable salt thereof, and fluticasone, or a pharmaceutically acceptable ester thereof, each in admixture with a pharmaceutically acceptable adjuvant, diluent or carrier.

19. A dry powder inhaler according to claim 16 characterised in that the adjuvant, diluent or carrier is selected from dextran, mannitol and lactose.

20. A dry powder inhaler according to claim 17 characterised in that the carrier is lactose.

21. A dry powder inhaler according to claim 17 characterised in that the dry powder inhaler is selected from those described in PCT/GB 00/04623.

22. A dry powder inhaler according to claim 17 characterised in that the dry powder inhaler is selected from those described in PCT/GB 00/03377.

23. A method according to claim 1 characterised in that the formoterol, or a pharmaceutically acceptable salt thereof, and fluticasone, or a pharmaceutically acceptable ester thereof, are administered by way of a nebuliser comprising a solution or a suspension of formoterol, or a pharmaceutically acceptable salt thereof, and fluticasone, or a pharmaceutically acceptable ester thereof.

24. A method according to claim 1 characterised in that a the amount of formoterol, or a pharmaceutically acceptable salt thereof, administered to a patient is from 20 to 500 μg and the amount of fluticasone, or a pharmaceutically acceptable ester thereof, administered to a patient is from 3 to 50 μg; once or twice daily.

25. A method according to claim 1 characterised in that the respiratory disorder is COPD.

26. A method according to claim 1 characterised in that the pharmaceutically acceptable salt of formoterol, is selected from an acid addition salts; hydrochloride, hydrobromide, sulphate, phosphate, maleate, tartrate, citrate, benzoate, 4-methoxybenzoate, 2- or 4-hydroxybenzoate, 4-chlorobenzoate, p-toluensulphonate, methanesulphonate, ascorbate, salicylate, acetate, fumarate, succinate, lactate, glutarate, gluconate, hydroxynaphthalenecarboxylate and oleate.

27. A method according to claim 26 characterised in that the pharmaceutically acceptable salt of formoterol, is the fumarate salt.

28. A method according to claim 1 characterised in that the pharmaceutically acceptable ester of fluticasone, is the propionate ester.

29. A method of attaining improved glucocorticoid receptor translocation into the nucleus by the administration of a therapeutically effective amount of a β₂ agonist and a steroid in therapeutically effective amounts wherein the method provides an improvement of at least 20% over prior art β₂ agonist and steroid combination therapies.

30. The use of formoterol, or a pharmaceutically acceptable salt thereof, in the manufacture of a medicament for use in the method according to claim 1.

31. The use of fluticasone, or a pharmaceutically acceptable ester thereof, in the manufacture of a medicament for use in the method according to claim 1.

32. The use of formoterol, or a pharmaceutically acceptable salt thereof, and fluticasone, or a pharmaceutically acceptable ester thereof, as active ingredients in the manufacture of a medicament to be administered separately, sequentially or simultaneously, provided that the active ingredients comprise separate compositions for the treatment or alleviation of a respiratory disorder.

33. The use of a glucocorticoid in the manufacture of a medicament with improved β₂ receptor expression.

34. A method according to claim 1 characterised in that the ratio of formoterol, or a pharmaceutically acceptable salt thereof, to fluticasone, or a pharmaceutically acceptable ester thereof, is in the range 1:0.4 to 1:167.

35. A method or an inhaler substantially as described with reference to the accompanying examples.
